(19) United States
(12) Patent Application Publication
(10) Pub. No.: US 2012/0104047 A1
(43) Pub. Date: May 3, 2012

(54) Composition for Relief of Insect Bites and Stings and Apparatus and Method for Its Delivery

(76) Inventor: Walter K. Lim, Rancho Santa Fe, CA (US)

(21) Appl. No.: 12/916,673

(22) Filed: Nov. 1, 2010

(51) Int. Cl. B67D 7/84; B67D 7/06; A61K 9/12; A61K 38/43; A61K 38/48

(52) U.S. Cl. 222/173; 424/94.1; 424/94.65; 424/43; 222/464.1; 222/409

(57) ABSTRACT

A composition to relieve itch, pain and swelling associated with a bite or sting, comprising a proteolytic enzyme dissolved in water, an anti-bacterial agent, and a pH lowering agent. The composition is in a dispensing container, and is devoid of other ingredients that may mask the effectiveness of the enzyme. In one form a liquid propellant floats on the composition and a dip tube extends from a discharge valve into the composition. In another form, a piston separates the composition from a pressurized gaseous propellant. In another form, the composition is in a bag in the container and pressurized gaseous propellant surrounds the bag. In a further form the bag is an elastic material that exerts pressure on the composition. In other forms the container is a single use bladder or pouch. The proteolytic enzyme preferably is a meat tenderizer such as Papain or Bromelain.

### TECHNICAL FIELD

This invention relates to compositions for topical application to relieve the itching, pain and swelling associated with insect bites and stings and other proteinaceous afflictions or irritations caused by any stinging jelly-like marine cnidarian, and to apparatus and method for delivery of the composition to the site of the bite or sting.

### BACKGROUND ART

Millions of people are bitten or stung each year by mosquitoes, bees, ants, flies, jelly fish, and the like. The great majority of such bites and stings merely produce discomfort, including pain, itching, and swelling at the site of the bite or sting, although in rare instances serious complications or even death can result. Mosquito bites, for instance, generally produce primarily itching and swelling at the site of the bite, although their bite can spread diseases such as Dengue fever and malaria. Bee stings generally produce almost immediate pain at the site of the sting, followed by itching and swelling, but persons especially susceptible to bee venom can experience much more serious symptoms and even death if not promptly treated. As used herein, the term “bees” is intended to include Vespids, i.e., wasps, yellow jackets and hornets. Jelly fish and ants, especially fire ants, generally produce symptoms similar to those experienced with bee stings.

Various remedies have been proposed in the prior art for relieving the symptoms associated with these bites and stings. Antihistamines have been used to suppress the histamine-induced wheal (swelling) and flare (vasodilation) response by blocking the binding of histamine to its receptors on nerves, vascular smooth muscle, glandular cells, endothelium, and mast cells. Although antihistamines can reduce the swelling and itching associated with insect bites and stings, they appear to have little effect on the pain caused by the stings and bites.

Compositions that contain proteolytic enzymes or proteases for relieving the itch, pain and swelling have been proposed for topical application to the affected area. Such compositions are disclosed, for example, in U.S. Pat. Nos. 5,543,149 and 7,166,281. These patents disclose the use of the proteolytic enzyme Papain, commonly used as a meat tenderizer.

Prior art remedies for relieving the itch, pain and swelling produced by bites and stings have met with limited success, perhaps due to the inclusion of other ingredients that dilute the active ingredient and/or inhibit its penetration into the skin of the patient. The literature has shown that chemicals such as alcohols and other solvents and oils are absorbed relatively slowly compared to water.

Further, the proteolytic activity or potency of the enzyme in meat tenderizer has a limited shelf life, particularly when it is in aqueous solution and/or exposed to oxygen.

U.S. Pat. No. 3,930,030, directed to a barbecue sauce with meat tenderizer, purports to maintain the potency of the proteolytic activity of Papain by maintaining the pH level of the composition between about pH 3.5 and pH 5.8, especially when the Papain is in aqueous solution

Applicant is not aware of any prior effective treatment for relieving the itch, pain and swelling resulting from insect and jelly fish bites and stings, wherein the treatment comprises a composition conveniently delivered as a spray onto the affected site from a dispensing container, and wherein the composition comprises a therapeutically effective amount of Papain or Bromelain dissolved in a suitable water based carrier, and further wherein the pH of the composition is adjusted to a level that preserves the potency of the proteolytic activity of the Papain or Bromelain. The literature has shown that water penetrates the skin fairly quickly, particularly in comparison with other chemicals such as alcohols and other solvents and oils which as noted above are absorbed relatively slowly. Placing the active enzyme in a water based carrier is an important feature of the present invention since the water and active enzyme carried thereby relatively quickly penetrate the skin and the enzyme can break down the toxins delivered by the bite or sting.

Accordingly, it would be desirable to have a composition that relieves the itch, pain and swelling associated with insect bites and stings, wherein the composition has an extended shelf life and wherein means is provided for quickly and easily applying the composition to an affected site.

### DISCLOSURE OF THE INVENTION

The present invention comprises a composition that relieves the itch, pain and swelling associated with insect bites and stings. The composition has an extended shelf life exceeding five years and means is provided for quickly and easily applying the composition to an affected site.

More particularly, the composition of the invention comprises a proteolytic enzyme as the active ingredient, dissolved in water as a carrier for the enzyme, and preferably a pH-lowering agent to lower the pH of the composition to a level to maintain the proteolytic activity of the enzyme in the aqueous solution.

In a preferred embodiment the proteolytic enzyme is Papain or Bromelain, commonly used as meat tenderizers, and an anti-bacterial preservative is in the composition to inhibit bacterial growth.

In a still further preferred embodiment, the pH lowering agent is citric acid, and the anti-bacterial agent is sodium benzoate, frequently used as a food preservative.

In yet a further preferred embodiment, water comprises about 98.81% of the composition, Papain or Bromelain comprises about 0.89%, citric acid comprises about 0.20%, and sodium benzoate comprises about 0.10% of the composition, and the pH level is maintained between about 3.5 and 5.8.

The composition preferably is placed in a dispensing container for discharging the composition as a spray against the affected site. In a preferred embodiment for personal use the container is small and has a narrow cross-section with a liquid propellant floated on top of the composition. In this preferred embodiment the container may be easily carried in a purse or pocket. The composition comprises about 97.7% of the contents of the container and the propellant comprises about 2.30%. The propellant preferably is pure grade isobutane. The propellant is not soluble in water and separates and floats on the surface of the composition. This lack of solubility is critical to performance of the invention. Moreover, there are no ingredients in the composition to cause emulsification. Compressed gas propellant systems using CO₂, N₂ or N₂O may also be used.

In a further preferred embodiment the container is a pressurized barrier pack that isolates the composition from the atmosphere so that the proteolytic enzyme is not rendered inactive by exposure to oxygen while in the container during storage.

In a still further preferred embodiment the discharge valve on the container is of the ball-in-valve type so that the container may be used in any orientation.

The container preferably is made of metal, preferably aluminum, but it may be made of other suitable materials such as plastic or glass. Lined steel cans are available but are not recommended for acidic products. The container and valve cup preferably are lined with a food grade coating of EPON™, available from the Miller-Stephenson Chemical Company of Danbury, Conn.

Although as illustrated and described herein pressure is exerted on the composition by a propellant in the container, it is possible that the pressure could be provided by a spring or by containing the composition in a stretchable elastomeric bladder, or the like.

Alternatively, the invention could be designed for one-time use, wherein the composition could be contained in a small pouch with an end that may be broken or cut off to dispense the composition onto the surface of the skin, or the composition could be contained in a foil pack or the like that can be torn or cut open in a conventional manner to dispense the composition.

In actual use of the invention to treat insect stings and bites, when the composition is applied to the site soon after the sting or bite occurs, effective relief is had in a matter of seconds. In instances of severe allergenic effects or reactions to insect bites and stings, it is important to apply the composition immediately after the sting or bite is received so that the enzymes in the active ingredient can quickly neutralize the toxins delivered by the bite or sting before they get into the bloodstream.

### BRIEF DESCRIPTION OF THE DRAWINGS

The foregoing, as well as other objects and advantages of the invention, will become apparent from the following detailed description when taken in conjunction with the accompanying drawings, wherein like reference characters designate like parts throughout the several views, and wherein:

FIG. 1 is a longitudinal sectional view of a first form of pressurized aerosol dispensing container according to the invention, wherein the container is tall and narrow, but relatively small, and a liquid propellant is floated on top of the product.

FIG. 2 is a longitudinal sectional view of a second form of pressurized aerosol dispensing container according to the invention, wherein the product is contained on one side of a piston inside the container and a pressurized gaseous propellant is on the other side of the piston.

FIG. 3 is a longitudinal sectional view of a third form of pressurized aerosol dispensing container according to the invention, wherein the product is contained in a bag inside the container and the bag is surrounded by a pressurized gaseous propellant and/or a compressed gas such as CO₂, for example, adsorbed on a material such as zeolite.

FIG. 4 is a greatly enlarged fragmentary view in longitudinal section of one type of ball-in-valve that may be incorporated in the discharge valve so that the container of the invention may be used in any orientation.

FIG. 5 depicts the ball-in-valve of FIG. 4 in its operative position to dispense product when the container is in an upright orientation.

FIG. 6 depicts the ball-in-valve of FIG. 4 in its operative position to dispense product when the container is in an inverted upside-down orientation.

FIG. 7 is a greatly enlarged fragmentary view in longitudinal section of another type of ball-in-valve that may be incorporated in the discharge valve so that the container of the invention may be used in any orientation, depicting the operative position of the ball-in-valve to dispense product when the container is in an upright orientation.

FIG. 8 depicts the ball-in-valve of FIG. 7 in its operative position to dispense product when the container is in an inverted upside-down orientation.

FIG. 9 is a perspective view of a single-use form of the invention wherein the composition is in a sealed bladder or pouch that is opened to dispense the composition.

FIG. 10 is a perspective view of a further form of single-use dispenser, wherein the composition is contained in a foil pack or the like that may be torn or cut open to dispense the composition.

FIG. 11 is a somewhat schematic longitudinal sectional view of another form of the invention wherein the composition is contained within a stretchable elastomeric bladder that exerts pressure on the composition and causes it to be dispensed when a discharge valve is opened.

### BEST MODES FOR CARRYING OUT THE INVENTION

The composition of the invention comprises a proteolytic enzyme as the active ingredient, water as a carrier, a pH lowering ingredient, and an anti-bacterial ingredient. The composition is free of creams, lotions or other materials that could reduce the effectiveness of the active ingredient when the composition is sprayed onto the skin of the person being treated. In a preferred embodiment, the commonly used meat tenderizer Papain or Bromelain is the active ingredient, dissolved in the water carrier, with citric acid added in an amount sufficient to lower the pH of the composition to a pH of between about 3.5 and about 5.8. Lowering of the pH to the indicated range preserves the proteolytic activity of the enzyme in the aqueous solution. Sodium benzoate is added in an amount sufficient to inhibit the growth of bacteria. Water comprises about 98.81% of the composition, Papain or Bromelain comprises about 0.89%, citric acid comprises about 0.20%, and sodium benzoate comprises about 0.10% of the composition.

The composition preferably is placed in a dispensing container for discharging the composition as a spray against the affected site.

A first and preferred form of container for use in the invention is shown at 20 in FIG. 1. The container 20 is relatively small, with a tall narrow configuration, preferably having a height of about 3 inches and a diameter of about ⅝ inch, for example, with a capacity of from about ½ ounce to about 2 ounces, for example. The composition C is disposed in the bottom portion of the container, and a liquid propellant P is floated on top of the composition. The composition is isolated from the atmosphere so that the proteolytic enzyme is not exposed to oxygen while in the container, which during storage would cause the enzyme to lose its proteolytic activity. The propellant preferably comprises a water insoluble hydrocarbon, preferably pure grade isobutane, with the liquid propellant comprising about 2.30% of the contents of the container and the composition comprising about 97.7%. As the composition is depleted from the container and the available free volume in the container consequently increases, some of the liquid propellant gasifies to restore pressure in the container. The container preferably is made of aluminum coated on its interior surface with a food grade liner such as EPON™, available from the Miller-Stephenson Chemical Company of Danbury, Conn. A discharge valve 14 on the upper end of the container is opened upon depression of an actuator button 15, enabling release from the container through dip tube 21 and discharge valve 14 of a desired quantity of the pressurized composition.

While the small container in the form of the invention described immediately above is especially suitable for personal use, larger containers in sizes up to about 20 ounces may be manufactured for use in the home or doctor's offices or hospitals and the like. The larger sizes would provide economy of scale in these environments.

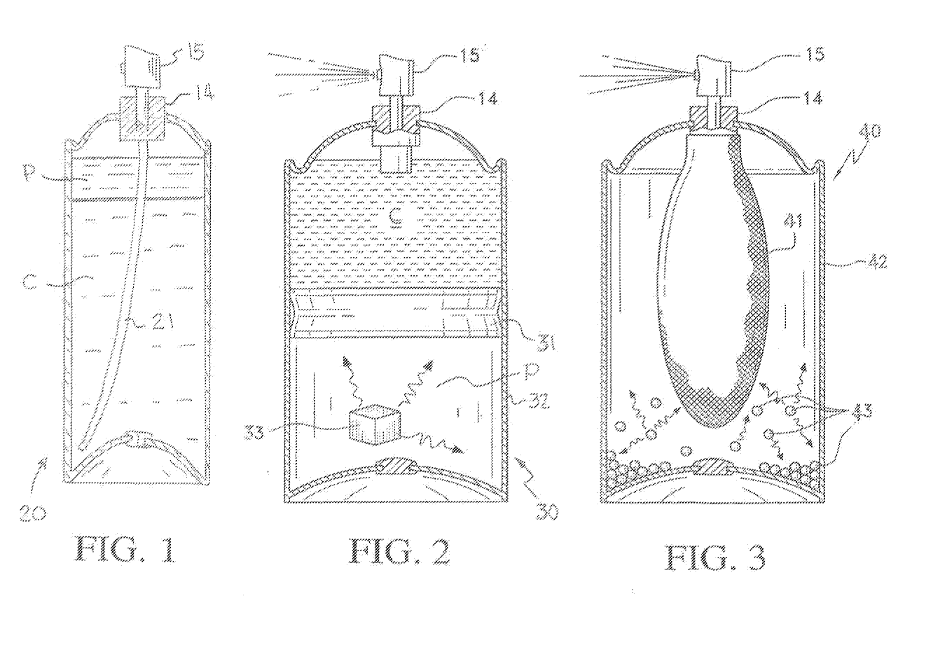

In a second form of the invention shown in FIG. 2, the container 30 is of substantially conventional construction and is of the barrier pack type, comprising a piston 31 in a can 32, with the piston 31 isolating the composition C from the propellant P. In this form of the invention the propellant is in pressurized gaseous form. If desired, a reserve supply of the gaseous propellant may be adsorbed on a sorbent material 33 such as activated carbon or zeolite, for example, for release of the adsorbed gas into the container to maintain the pressure at a predetermined level as product is depleted, as described more fully in applicant's copending patent application Ser. No. 11/250,235, filed Oct. 14, 2005, the disclosure of which is incorporated in full herein by reference. Since there is no dip tube extending to the bottom of the container and the composition C is confined to the space immediately adjacent the discharge valve 14 in this form of the invention, the container 30 may be operated in any orientation. The container in this form of the invention also preferably is made of aluminum coated on its interior surface with a food grade liner such as EPON™, and as in the previous form of the invention the discharge valve 14 is opened to enable pressurized discharge of the composition C upon depression of actuator button 15.

A third form of dispenser that may be used in the invention is shown at 40 in FIG. 3. In this form of the invention the container also is of the barrier pack type, comprising a bag 41 secured in the can 42 in a conventional manner, with a discharge valve 14 operated by depression of an actuator button 15. The composition C is confined inside the bag, and a pressurized gaseous propellant P is in the space between the bag and the can. Since there is no dip tube extending to the bottom of the container and the composition C is confined to the space immediately adjacent the discharge valve 14 in this form of the invention, the container 40 may be operated in any orientation. The container in this form of the invention also preferably is made of aluminum coated on its interior surface with a food grade liner such as EPON™, and as in the previous form of the invention the discharge valve 14 is opened to enable pressurized discharge of the composition C upon depression of actuator button 15. If desired, a reserve supply of the gaseous propellant may be adsorbed on a sorbent material 43 such as activated carbon or zeolite, for example, for release of the adsorbed gas into the container to maintain the pressure at a predetermined level as product is depleted, as described more fully in applicant's copending patent application Ser. No. 11/250,235, filed Oct. 14, 2005, the disclosure of which is incorporated in full herein by reference.

If desired, the container 20 of FIG. 1 may be designed to operate in any orientation by incorporation in the discharge valve 14 of a valve mechanism operative to enable flow from the dip tube 21 when the container is an upright orientation but to block flow from the dip tube and enable flow from a secondary inlet when the container is in an inverted upside down orientation.

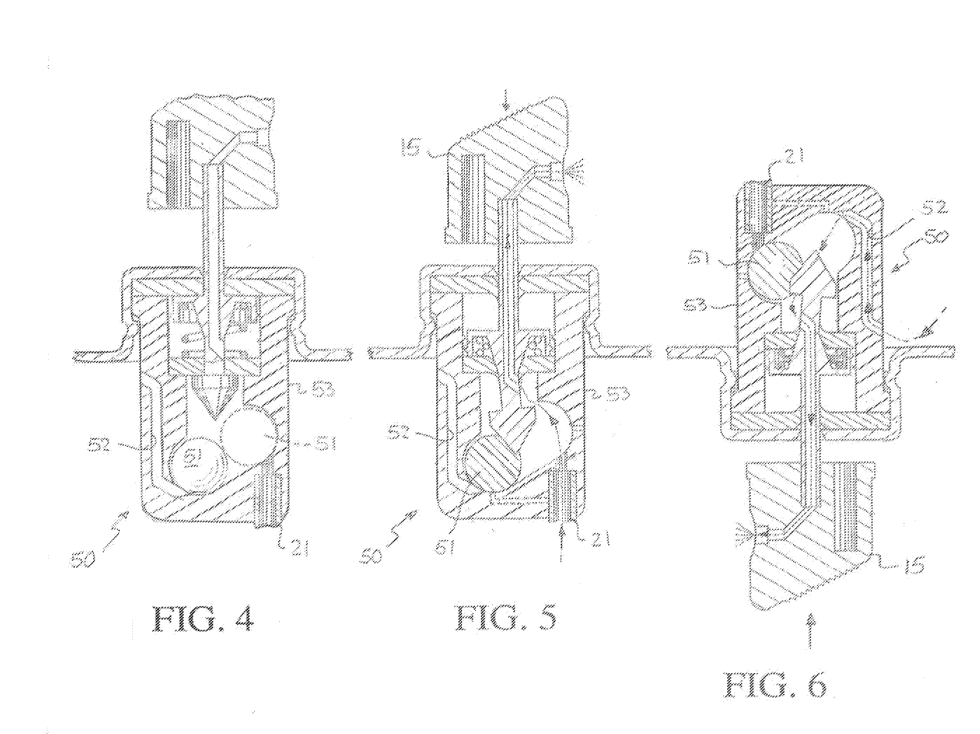

One such valve mechanism 50 is shown in FIGS. 4-6, wherein a ball 51 is caused by gravity to move into a position blocking flow from the secondary inlet 52 in the valve housing 53 but to enable flow from the dip tube 21 when the container is in an upright orientation as depicted in FIGS. 4 and 5, or to block flow from the dip tube and enable flow through the secondary inlet 52 when the container is in an inverted orientation as depicted in FIG. 6. It will be noted that ball 51 is held in its respective operative positions upon actuation of the discharge valve, thereby preventing pressure in the container from moving the ball. The valve mechanism 50 is more fully described and illustrated in U.S. Pat. No. 4,978,038, the disclosure of which is incorporated in full herein by reference.

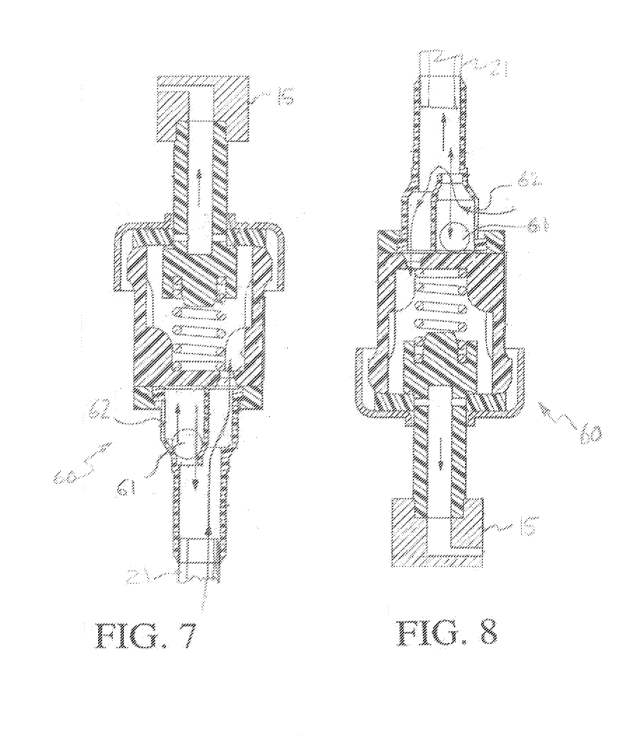

FIGS. 7 and 8 depict another valve mechanism 60 that could be used in the container 20 of FIG. 1 to enable the container to be operated in any orientation. This form of valve functions similarly to the embodiment shown in FIGS. 4-6 in that a ball 61 moves by gravity between a first position blocking flow from a secondary inlet 62 and enabling flow from dip tube 21, and a second position enabling flow through the secondary inlet. This valve mechanism 60 is more fully described and illustrated in U.S. Pat. No. 5,803,319, the disclosure of which is incorporated in full herein by reference.

It should be understood that the two valve mechanisms 50 and 60 described above and the piston or bag-in-a-can barrier packs are examples of mechanisms that could be used to enable the container 20 to be operated in any orientation, but other means may be suitable.

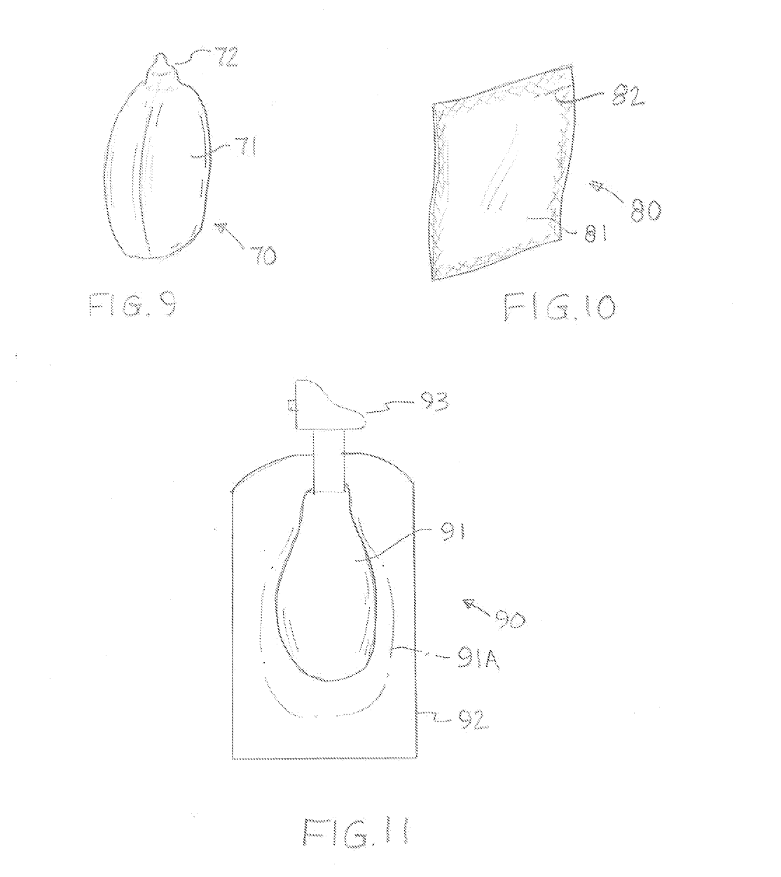

A form of the invention intended for single use is shown at 70 in FIG. 9. In this form of the invention the composition is sealed in a small pouch 71 with an end 72 that may be cut or broken off to enable discharge of the composition. If desired, the pouch may be made squeezable so that it is squeezed to dispense the composition.

Another form of single-use dispenser is shown at 80 in FIG. 10, wherein the composition is contained in a foil pack or the like 81 of substantially conventional construction, preferably with a frangible area 82 to facilitate opening of the pack and dispensing of the composition.

A further form of the invention is shown at 90 in FIG. 11. In this form of the invention the composition is pumped under pressure into a stretchable elastomeric bladder 91, expanding the bladder to a stretched position indicated by the broken line 91A. When the discharge valve 93 is opened the tendency of the bladder to return to its unstretched condition causes the bladder to exert pressure on the composition and force it through the discharge valve 93.

It should be noted that preventing exposure of the proteolytic enzyme to atmospheric oxygen, and lowering the pH of the composition, are important considerations in the present invention in order to maintain the effectiveness of the proteolytic enzyme and prolong the shelf life of the composition. Further, the active ingredient in the composition of the invention is able to more readily penetrate the skin of a person using the product because there are no unnecessary ingredients in the composition that could dilute the active ingredient and/or mask it against penetration into the skin. Moreover, the propellant is not emulsified into the product, which could cause the product to foam and reduce its ability to quickly penetrate the skin.

Actual use of the composition of the invention by spraying it onto the skin proximate to the affected site has demonstrated its effectiveness in almost immediately relieving the pain and itching associated with insect bites and stings and in relieving more serious allergic reactions that can result from bites and stings. The composition does not contain any emollients or anti-irritants or moisturizers or the like that would inhibit penetration of the enzyme into the skin of the user and reaction of the enzyme with the toxins.

The meat tenderizers Papain and Bromelain have been described herein as sources for the active enzyme to treat bites and stings, but it should be understood that other enzymes having suitable properties may be used, such as subtilisin or pancreatin, for example.

Although particular embodiments of the invention are illustrated and described in detail herein, it is to be understood that various changes and modifications may be made to the invention without departing from the spirit and intent of the invention as defined by the scope of the appended claims.

What is claimed is:

1. A composition for topical application to the skin of a person proximate the site of a bite or sting to relieve the itch, pain and swelling associated with the bite or sting, said composition comprising:
an effective amount of a proteolytic enzyme dissolved in water;
an anti-bacterial agent; and
a pH lowering agent.

2. The composition as claimed in claim 1, wherein:
the proteolytic enzyme is Papain.

3. The composition as claimed in claim 1, wherein:
the proteolytic enzyme is Bromelain.

4. The composition as claimed in claim 1, wherein:
the anti-bacterial agent is sodium benzoate.

5. The composition as claimed in claim 1, wherein:
the pH lowering agent is citric acid.

6. The composition as claimed in claim 1, wherein:
the proteolytic enzyme is Papain;
the anti-bacterial agent is sodium benzoate; and
the pH lowering agent is citric acid.

7. The composition as claimed in claim 1, wherein:
the proteolytic enzyme is Bromelain;
the anti-bacterial agent is sodium benzoate; and
the pH lowering agent is citric acid.

8. The composition as claimed in claim 6, wherein:
said water comprises about 98.81% of the composition;
said Papain comprises about 0.89% of the composition;
said citric acid comprises about 0.20% of the composition; and
said sodium benzoate comprises about 0.10% of the composition.

9. The composition as claimed in claim 7, wherein:
said water comprises about 98.81% of the composition;
said Bromelain comprises about 0.89% of the composition;
said citric acid comprises about 0.20% of the composition; and
said sodium benzoate comprises about 0.10% of the composition.

10. In combination, a composition for relieving the itching, pain and swelling associated with an insect bite or sting, and a container holding the composition for dispensing it onto the skin of a person proximate the site of the bite or sting, wherein:
the container has an interior normally sealed from the atmosphere, and means for opening the container so that a quantity of the composition can be dispensed from the container onto the skin of a person proximate the site of the bite or sting; and
the composition comprises an aqueous solution of a proteolytic enzyme dissolved in water, an anti-bacterial agent, and a pH-lowering agent.

11. The combination as claimed in claim 10, wherein:
the container comprises a can;
a propellant is in the can exerting a predetermined pressure on the composition; and
the means for opening the container comprises a normally closed discharge valve that may be opened to permit discharge under pressure of a quantity of said composition.

12. The combination as claimed in claim 11, wherein:
the propellant is a liquid propellant floating on top of the composition, a portion of said liquid propellant gasifying when the pressure in the can falls below said predetermined pressure to restore said predetermined pressure in the can; and
a dip tube extends from the discharge valve into the composition to enable flow of the composition from the can through the discharge valve when the valve is opened.

13. The combination as claimed in claim 11, wherein:
said composition is in an upper portion of said can;
said propellant is a pressurized gaseous propellant in a lower portion of the can; and
a piston separates the propellant from the composition, said pressurized gaseous propellant urging said piston toward said discharge valve to force said composition through said discharge valve when the valve is opened.

14. The combination as claimed in claim 11, wherein:
said composition is confined in a bag in the can, said bag being attached at one end to said discharge valve with the interior of the bag in fluid communication with the discharge valve; and
said propellant is a pressurized gaseous propellant surrounding the bag to exert pressure on the bag and thereby exert pressure on the composition contained in the bag to force the composition through the discharge valve when the valve is opened.

15. The combination as claimed in claim 10, wherein:
the container comprises a small one-time use sealed bladder having a portion that may be opened to enable said composition to be dispensed from the bladder.

16. The combination as claimed in claim 10, wherein:
the container comprises a sealed foil pack that may be opened to dispense the composition from the foil pack.

17. The combination as claimed in claim 12, wherein:
said dip tube is connected with a primary opening through said discharge valve for flow of said composition through said dip tube and primary opening when the can is in an upright orientation;
said discharge valve has a secondary opening in communication with an upper portion of the interior of the can adjacent said discharge valve for flow of said composition into and through said discharge valve when the can is in an inverted upside down orientation; and
a ball valve is in said discharge valve operable to block flow of said composition through said secondary opening but permit flow from said dip tube and through said primary opening when the can is in an upright orientation and to block flow from said dip tube but permit flow through said secondary opening when said container is in an inverted upside down orientation, whereby said container may be operated in any orientation.

18. The combination as claimed in claim 10, wherein:
the container comprises a can;
the means for opening the container comprises a normally closed discharge valve that may be opened to permit discharge under pressure of a quantity of said composition; and
the composition is contained under pressure in a stretchable elastomeric bladder that is connected at one end to the discharge valve and is stretched to an expanded position by the pressurized composition, and when the discharge valve is opened the tendency of the bladder to return to its unstretched condition causes the bladder to exert pressure on the composition and force it through the discharge valve.
